(19) United States
(12) Patent Application Publication
(10) Pub. No.: US 2012/0004404 A1
(43) Pub. Date: Jan. 5, 2012

(54) RADIOLABELLING METHODS

(76) Inventors: Edward George Robins, London (GB); Erik Arstad, London (GB)

(21) Appl. No.: 13/256,483

(22) PCT Filed: Mar. 15, 2010
(86) PCT No.: PCT/US2010/027278

§ 371 Date: Sep. 14, 2011

Related U.S. Application Data
(60) Provisional application No. 61/164,964, filed on Mar. 31, 2009.

(30) Foreign Application Priority Data
Mar. 31, 2009 (GB) 0905515.3

(51) Int. Cl. C07H 1/00; C07C 209/36

(52) U.S. Cl. 536/28.54; 564/417

(57) ABSTRACT

The present invention relates to the field of [¹⁸F] radiofluorination chemistry for the preparation of Positron Emission Tomography (PET) radioligands and [¹⁸F] radiofluorinating reagents. The invention further provides kits for preparation of the same.

The present invention relates to the field of [¹⁸F]radiofluorination chemistry for the preparation of Positron Emission Tomography (PET) radioligands and [¹⁸F]radiofluorinating reagents. The invention further provides kits for preparation of the same.

Commonly used methods for introducing ¹⁸F are either direct displacement of a leaving group by nucleophilic [¹⁸F]Fluoride, or using electrophilic reagents such as [¹⁸F]F₂, [¹⁸F]acetylhypofluorite (Lerman et al, Appl. Radiat. Isot. 49 (1984), 806-813) or N-[¹⁸F]fluoropyridinium salt (Oberdorfer et al, Appl. Radiat. Isot. 39 (1988), 806-813), or by a two step process involving preparation of an ¹⁸F radiofluorinated labelling reagent which is in turn reacted with a ligand precursor by a second reaction such as an alkylation. This latter approach generally involves incorporating via a nucleophilic centre O, N, or S, which in turn can lead to metabolic instability of the resulting PET radioligand. Furthermore, the value of PET is the ability to use a radioligand which closely mimics the structure of the therapeutic pharmacaphore and it is therefore not always desirable to incorporate O, N, or S into the PET radioligand.

Steiniger et al J. Labelled Compounds and Radiopharmaceuticals 49(9), 817-827 (2006) describes coupling of certain aryl boronic acids with 4-[¹⁸F]fluoroiodobenzene to form 4-[¹⁸F]fluorobiphenyl compounds. Similar couplings have been used in the field of ¹¹C-labelling, particularly for formation of [¹¹C]tolyl derivatives, for example Hoestler et al, J. Labelled Compounds and Radiopharms (2005), 48, 629-634. Hoestler et al, J. Org. Chem. (1998), 63, 1348-1351 describes coupling of [11C]methyl iodide with an alkyl borane.

However, there still exists a need for alternative [¹⁸F]radiofluorinating reagents or synthons and [¹⁸F]radiofluorination methodologies, which allow rapid, chemoselective introduction of an [¹⁸F] label into biomolecules, under mild conditions to give [¹⁸F]-labelled products in high radiochemical yield and purity.

Additionally, there is a need for such methodologies which are amenable to automation to facilitate preparation of [¹⁸F]radioligands in the clinical setting. The methods described herein provide for direct [¹⁸F]fluoroalkylation to provide biomolecules that may otherwise be unavailable.

According to one aspect of the invention, there is provided a method for the preparation of a compound of formula (I):

Y-(C₁₋₈alkyl)-¹⁸F (I)

wherein Y is a biological targetting moiety,
which comprises:
reaction of a compound of formula (II):

Y-B(Z)₂ (II)

wherein Y is as defined for the compound of formula (I), B is boron, and Z is selected from hydroxy, C₁₋₆alkoxy, C₁₋₆alkyl, C₅₋₁₂aryloxy and C₅₋₁₂aryl and each Z is optionally substituted by 1 to 4 substituents selected from hydroxy, C₁₋₆alkyl, C₁₋₆alkoxy, and halo, or both groups Z together with the B to which they are attached form an organoboron cyclic moiety;
with a compound of formula (III) :

X-(C₁₋₈alkyl)-¹⁸F (III)

wherein X is chloro, bromo, iodo, a C₁₋₆alkylsulphonate, haloC₁₋₆alkylsulphonate, or arylsulphonate (such as trifluoromethanesulphonate, methanesulphonate, tolylsulphonate) ; and the C₁₋₈alkyl group is as defined for the compound of formula (I);
in a suitable solvent, and in the presence of a base and a transition metal catalyst.

In the compounds of formulae (I) and (III), Y is a biological targeting moiety, suitably a non-peptide small drug-like molecule or a protected derivative thereof, typically a substituted or unsubstituted, aromatic or aliphatic 5 to 8 membered monocyclic ring, or a 10 to18 membered fused or unfused bicyclic ring system comprised of carbon, hydrogen, and optionally one to six heteroatoms selected from oxygen, nitrogen, and sulphur.

The C₁₋₈alkyl group in the compounds of formulae (I) and (III) is a straight or branched chain alkyl group or a cyclic alkyl group, suitably selected from methyl, ethyl, iso-propyl, n-propyl, n-butyl, cyclohexyl, and cyclooctyl.

In the compound of formula (II), the term organoboron cyclic moiety means a C₄₋12 mono or bicyclic aliphatic hydrocarbyl group further containing boron, such as 9-borabicyclo[3.3.1]nonyl or a C₅₋₁₂ mono or bicyclic aryl group further containing boron, such as

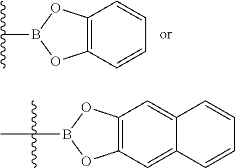

wherein the aryl rings may optionally be substituted by 1 to 4 substituents selected from hydroxy, C₁₋₆alkyl, C₁₋₆alkoxy, and halo.

In the compound of formula (II) , Z is suitably selected from hydroxy, methoxy, ethoxy, methyl, and ethyl or the group —B(Z)₂ is 9-borabicyclo[3.3.1]nonyl,

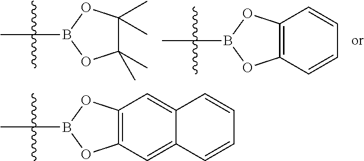

wherein the aryl rings may optionally be substituted by 1 to 4 substituents selected from hydroxy, C₁₋₆alkyl, C₁₋₆alkoxy, and halo.

X in the compound of formula (111) is more suitably bromo or iodo, most suitably bromo.

In one aspect of the invention, the compound of formula (III) is selected from ¹⁸F-CH₂Br, ¹⁸F-CH₂CH₂Br and ¹⁸F-CH₂CH₂CH₂Br.

Suitable solvents include N,N-dimethylformamide, dimethylsulphoxide, dichloromethane, chloroform, acetonitrile, toluene, tetrahydrofuran, iso-propanol, tert-amyl alcohol , diethyl ether, and tetrahydrofuran.

The transition metal catalyst is suitably a palladium or nickel catalyst. Preferred nickel catalysts include nickel amino alcohol derivatives such as Nil₂/trans-2-aminocyclohexanol or NiCl₂.Glyme/Prolinol, nickel metal (in the form of a finely divided powder, or nickel reaction vessel). Suitable Pd catalysts include Pd(PPh₃)₂Cl₂, Pd(PPh₃)₄, Tris(dibenzylideneacetone)dipalladium (Pd₂(dba)₃), Pd₂(dba)₃)/P(cyclohexyl)₃, Pd ₂(dba)₃/lPrHCl where lPr=1,3-bis(2,6-diisopropylphenyl)imidazol-2-ylidene, [1,1′-Bis(diphenylphosphino)ferrocene]dichloropalladium(II) (Pd(dppf)Cl₂), Pd(OAc)₂/P(t-Bu)₂Me, Pd(OAc)₂/P(cyclohexyl)₃.

The method is suitably performed at a non-extreme temperature, suitably at ambient temperature or elevated temperature up to the boiling point of the solvent, for example up to 100° C. In one aspect of the invention, the method is performed using microwave heating.

The reaction comprises a base, suitably an inorganic base such as potassium carbonate, caesium carbonate, sodium hydroxide, caesium hydroxide, tripotassium phosphate, or a Lewis Base such as KOt-Butyl.

Compounds of formula (II) may be prepared by methods well known to the person skilled in the art, for example as described in Miyaura et al, Chem Rev 1995, vol 95(7); Brown et al, Organometallics (1983), 2, 1311-1316; Yang et al, Medicinal Research Reviews, Vol 23(3), 346-368 (2003); Coord Chem Rev 2002, 224(1-2), 171-243; and Boronic Acids—Preparation and Applications in Organic Synthesis, (Wiley-VCH, 2006) by Dennis G. Hall.

Compounds of formula (III) may be prepared from commercially available starting materials by methods which are well known in the art. For example, [¹⁸F]Fluorohaloalkanes have previously been prepared by nucleophilic displacement, by [¹⁸F]F⁻, of a leaving group from a suitable precursor compound. Thus, for example Zhang et al, Applied Radiation and Isotopes 57, 335-342 (2002), describes synthesis of [¹⁸F]fluoroethyl bromide by nucleophilic displacement of 2-trifluoromethanesulphonyloxy ethylbromide with [¹⁸F]F⁻ and Seung-Jun et al Applied Radiation and Isotopes (1999), 51, 293-7 describes an analogous synthesis of 3-[¹⁸F]fluoropropylbromide. A similar method is described in Comagic et al Applied Radiation and Isotopes (2002), 56, 847-851 wherein 2-bromo-1-[¹⁸F]fluoroethane is prepared by nucleophilic displacement of 1,2-dibromoethane with [¹⁸F]F⁻. Alternative methods for synthesis of [¹⁸F]fluorohaloalkanes may be found in WO2004/029006. Other compounds of formula (III) may be prepared by analogy to the methods of for example: J. Med. Chem., 1991, 34(4), 1363; J. Med. Chem., 1996, 36(26), 5110; and JLCR 2001, 44, S909-S911.

Typical precursor compounds which may be [¹⁸F]fluorinated to provide a compound of formula (III) include those of formula (IV):

X-(C₁₋₈alkyl)-L (IV)

wherein X is chloro, bromo, iodo, a C₁₋₆alkylsulphonate, haloC₁₋₆alkylsulphonate, or arylsulphonate (such as trifluoromethanesulphonate, methanesulphonate, tolylsulphonate); the C₁₋₈alkyl group is as defined for the compound of formula (I); and L is a leaving group, for example, selected from chloro, bromo, iodo, a C₁₋₆alkylsulphonate, haloC₁₋₆alkylsulphonate, or arylsulphonate (such as trifluoromethanesulphonate, methanesulphonate, tolylsulphonate).

[¹⁸F]fluoride is conveniently prepared from ¹⁸O-enriched water using the (p,n)-nuclear reaction, (Guillaume et al, Appl. Radiat. Isot. 42 (1991) 749-762) and generally isolated as the potassium salt which is dried and solubilised with a phase transfer agent such as a tetraalkylammonium salt or an aminopolyether (for example, Kryptofix 2.2.2).

As would be appreciated by a person skilled in the art, protecting groups may be required during synthesis of a compound of formula (I) to prevent unwanted side-reactions. Therefore, protected derivatives of synthetic intermediates such as a compound of formula (II) comprise one or more protecting groups to prevent unwanted reaction of certain reactive groups. Suitable protecting groups may be found in Protecting Groups in Organic Synthesis, Theodora W. Greene and Peter G. M. Wuts, published by John Wiley & Sons Inc. which describes methods for incorporating and removing such protecting groups.

Conveniently, the compound of formula (II) could be provided as part of a kit to a radiopharmacy. The kit may comprise a cartridge which can be plugged into a suitably adapted automated synthesiser. The cartridge may contain, apart from the compound of formula (II), a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customers requirements for radioactive concentration, volumes, time of delivery etc. Conveniently, all components of the kit are disposable to minimise the possibilities of contamination between runs and may be sterile and quality assured.

The invention further provides a radiopharmaceutical kit for the preparation of a compound of formula (I) as defined above for use in PET, which comprises:
(i) a vessel containing a compound of formula (II) as defined above; and
(ii) a vessel containing a compound of formula (IV) as defined above and means for contacting said compound of formula (IV) with a source of ¹⁸F⁻.

### EXAMPLES

### ¹⁸F-fluoroalkylation of aryl boronic acids

[¹⁸F]Fluoroalkylation using Ni-catalysed Suzuki cross-coupling chemistry offers a route to the direct insertion of labelling agents of the type 1-X-(CH₂)ₙ-¹⁸F (such that X=I, Br) with boronic acids. The recently reported serotonin transporter ligands [¹⁸F]AFM, [¹⁸F]AFE and [¹⁸F]AFP, described by Y. Huang et al. (J. Med. Chem., 2005, 48, 2559), were labelled by nucleophilic displacement of chloride or tosylate leaving groups with [¹⁸F]fluoride and subsequent reduction of the aryl nitro group. Application of the Ni-catalysed Suzuki cross-coupling chemistry would facilitate the coupling of a variety of [¹⁸F]fluoroalkyl groups using a common boronic acid precursor prior to nitro group reduction.

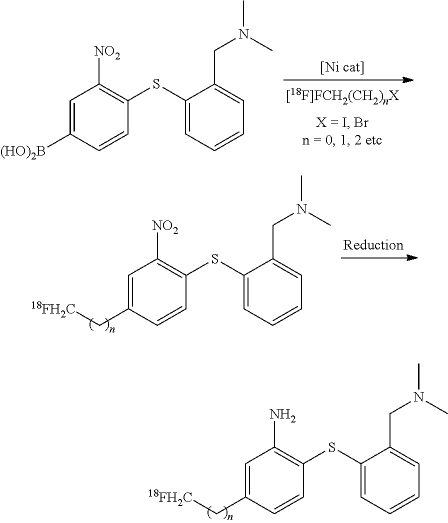

### Example 1

### Synthesis of [¹⁸F]AFE(2[[2-Amino-4-(2-[¹⁸F]fluoroethyl)phenyl[thio]-N,N-dimethylbenzenemethanamine)

### Step 1

### Synthesis of the boronic acid precursor [2-(4-boronic acid-2-nitro-phenylsulfanyl)-benzyl]-dimethyl-amine.

Starting from 2-thio-N,N-dimethylbenzamide, reaction with 1-bromo-4-iodo-2-nitrobenzene in the presence of potassium carbonate base according to the method of Choi et al. (Journal Label. Compd. Radiopharm., 2001, 44, S190-192) yields 2-(4-iodo-2-nitro-phenylsulfanyl)-N,N-dimethyl-benzamide. Subsequently, reduction of the benzamide with borane yields [2-(4-iodo-2-nitro-phenylsulfanyl)-benzyl]-dimethyl-amine. Reaction of the iodide at low temperature (−78° C.) in anhydrous solvent such as tetrahydrofuran with either an alkyl lithium reagent (for example n-BuLi) or with a Grignard reagent such as isopropyl magnesium bromide followed by quenching with a trialkyborate (e.g. triisopropylborate) and aqueous acid work up provides the bornic acid derivative [2-(4-boronic acid-2-nitro-phenylsulfanyl)-benzyl]-dimethyl-amine.

### Step 2

### Suzuki Coupling Chemistry to Prepare [¹⁸F]AFE, (2-[[2-Amino-4-(2-[¹⁸F]fluoroethyl)phenyl]thio]-N,N-dimethylbenzenemethanamine)

Reaction of [2-(4-boronic acid-2-nitro-phenylsulfanyl)-benzyl]-dimethyl-amine with [¹⁸F]fluoroethyl bromide in a polar solvent (such as tetrahydrofuran, dioxane) in the presence of a suitable transition metal catalyst (e.g. Nil₂/trans-2-aminocyclohexanol) and base (e.g. potassium phosphate) at room temperature or at higher yields the desired cross-coupling product. For the purpose of this example, [¹⁸F]Fluoroethyl bromide could be prepared according to the published procedure of Bauman et al.(Tetrahedron Lett., 2003, 44, 9165). To complete the synthesis of [¹⁸F]AFE, reduction of the nitro group is achieved in an analogous way to that described by Y. Huang et al. (J. Med. Chem., 2005, 48, 2559) through treatment of the nitro compound by Cu(OAC)₂ or SnCl₂ catalysed sodium borohydride redution of the nitro group to the corresponding amine.

### ¹⁸F-fluoroalkvlation of vinyl boronic acids

Synthesis of the radiolabelled nucleoside 5-(2-[¹⁸F]fluoroethyl)-2′-deoxyuridine, [¹⁸F]FEDU has recetly been reported by C.-S. Yu et al. (J. Label. Compd. Radiopharm. 2003, 46, 421) and this radiotracer was radiolabelled by nucleophilic substitution of a tosylate leaving group. Ni-catalysed cross-coupling of the 5-boronic acid derivative with [¹⁸F]fluoroethylbromide should furnish the desired [¹⁸F]fluoroethyl labelled, O-protected tracer.

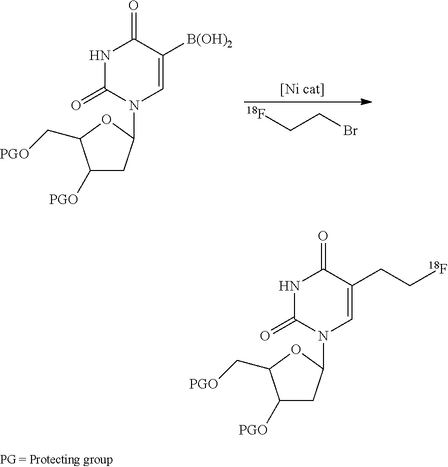

What is claimed is:

1. A method for the preparation of a compound of formula (I):
Y-(C₁₋₈alkyl)-¹⁸F (I)
wherein Y is a biological targetting moiety,
which comprises:
reaction of a compound of formula (II):
Y-B(Z)₂ (II)
wherein Y is as defined for the compound of formula (I), B is boron, and Z is selected from hydroxy, C₁₋₆alkoxy, C₁₋₆alkyl, C₅₋₁₂aryloxy and C₅₋₁₂aryl and each Z is optionally substituted by 1 to 4 substituents selected from hydroxy, C₁₋₆alkyl, C₁₋₆alkoxy, and halo, or both groups Z together with the B to which they are attached form an organoboron cyclic moiety;
with a compound of formula (III):
X-(C₁₋₈alkyl)-¹⁸F (III)
wherein X is chloro, bromo, iodo, a C₁₋₆alkylsulphonate, haloC₁₋₆alkylsulphonate, or arylsulphonate; and the C₁₋₈alkyl group is as defined for the compound of formula (I);
in a suitable solvent, and in the presence of a base and a transition metal catalyst.

2. A method according to claim 1 wherein in the compound of formula (II), Z is selected from hydroxy, methoxy, ethoxy, methyl, and ethyl or the group —B(Z)₂ is 9-borabicyclo[3.3.1]nonyl, 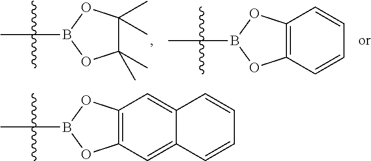
wherein the aryl rings may optionally be substituted by 1 to 4 substituents selected from hydroxy, C₁₋₆alkyl, C₁₋₆alkoxy, and halo.

3. A method according to claim 1 wherein in the compound of formula (III), X is bromo or iodo.

4. A method according to claim 1 wherein the compound of formula (III) is selected from ¹⁸F—CH₂Br, ¹⁸F—CH₂CH₂Br and ¹⁸F—CH₂CH₂CH₂Br.

5. A radiopharmaceutical kit for the preparation of a compound of formula (I) as defined in claim 1 for use in PET, which comprises:
(i) a vessel containing a compound of formula (II) as defined in claim; and
(ii) a vessel containing a compound of formula (IV):
X-(C₁₋₈alkyl)-L (IV)
wherein X is chloro, bromo, iodo, a C₁₋₆alkylsulphonate, haloC₁₋₆alkylsulphonate, or arylsulphonate; the C₁₋₈alkyl group is as defined for the compound of formula (I); and L is a leaving group selected from chloro, bromo, iodo, a C₁₋₆alkylsulphonate, haloC₁₋₆alkylsulphonate, or arylsulphonate;
and means for contacting said compound of formula (IV) with a source of ¹⁸F⁻

6. A method according to claim 1 wherein in the compound of formula (III), X is bromo.

7. A method according to claim 1 wherein said solvent is N,N-dimethylformamide, dimethylsulphoxide, dichloromethane, chloroform, acetonitrile, toluene, tetrahydrofuran, iso-propanol, tert-amyl alcohol, diethyl ether, or tetrahydrofuran.

8. A method according to claim 1 wherein said transition metal catalyst is a palladium or nickel catalyst.

9. A method according to claim 1 wherein said transition metal catalyst is a nickel catalyst.

10. A method according to claim 9 wherein said nickel catalyst is a nickel amino alcohol derivative, Nil2/trans-2-aminocyclohexanol or NiCl2.Glyme/Prolinol, or nickel metal in the form of a finely divided powder, or a nickel reaction vessel.

11. A method according to claim 1 wherein said transition metal catalyst is a palladium catalyst.

12. A method according to claim 11 wherein said palladium catalyst is Pd(PPh₃)₂Cl₂ , Pd(PPh₃)₄, Pd₂(dba)₃, Pd ₂(dba)₃)/P(cyclohexyl)₃, Pd₂(dba)₃/IPrHCl where IPr is 1,3-bis(2,6-diisopropylphenyl)imidazol-2-ylidene, Pd(dppf)Cl₂, Pd(OAc) ₂/P(t-Bu)₂Me, or Pd(OAc)₂/P(cyclohexyl)₃.
